(19) United States
(12) Patent Application Publication
(10) Pub. No.: US 2023/0334124 A1
(43) Pub. Date: Oct. 19, 2023

(54) Automated Behavioural Monitoring Unit

(71) Applicant: TBIAS Pty Ltd, Mermaid Beach (AU)

(72) Inventor: Adam Carroll, Mermaid Beach (AU)

(21) Appl. No.: 17/768,348

(22) PCT Filed: Apr. 29, 2020
(86) PCT No.: PCT/AU2020/050422

§ 371 Date: Apr. 12, 2022

(30) Foreign Application Priority Data
Oct. 14, 2019 (AU) 2019903863
Apr. 29, 2020 (WO) PCT/AU2020/050422

(51) Int. Cl. G06F 18/26; G06Q 50/26

(52) U.S. Cl. CPC G06F 18/26; G06Q 50/265

(57) ABSTRACT

An automated behavioral monitoring unit comprising one or more sensors, a processor in the unit connected to the sensors, an object library stored on the unit and an operating program. The program is adapted to recognize and classify objects detected by the sensors by accessing the object library stored on the unit, track the orientation of the objects with respect to each other, identify spatial orientations of the objects and people which are abnormal and communicate details of abnormalities to at least one remote user device. No imagery from the sensors is transmitted from the unit to the remote user device in order to safeguard the privacy of people being monitored.

### TECHNICAL FIELD

The present invention relates to the surveillance industry and, more particularly to a system capable of automatically monitoring a private place without violating people's privacy.

### BACKGROUND

Surveillance systems are available to monitor the behaviour of people or objects in public places. However, these systems have limited application in private places because they transmit camera footage to third parties. Camera footage can violate the privacy of people. Camera footage can be distributed online, which can further violate the privacy of people.

There are many places where it would be beneficial to monitor people in private places, but it cannot be practically implemented because of privacy concerns. These include private places such as aged care facilities, hospitals, asylums, schools, public toilets, prison rooms and private homes.

Aged care facilities may currently use camera systems to watch for abnormal behaviour such as falls, spillages, obstacles, or escape attempts. However, human employees are required to watch the footage from each camera and raise the alarm if abnormal behaviour is observed. Such camera systems can be expensive to install and expensive to monitor in terms of staff wages. It can be practically difficult or impossible for a single person to monitor an aged facility with many rooms because of both wages and technology costs.

Video monitoring systems for babies are not effective unless a parent is visually monitoring camera footage of the baby. The video monitoring cannot alert the parent that a baby is lying on its face, for example, unless the parent is looking at the monitor at the time the incident occurs.

There is a need for a system which is adapted to automatically monitor a private place but does not violate people's privacy.

The object of the present invention is to overcome or at least substantially ameliorate the aforementioned problems.

### SUMMARY OF THE INVENTION

According to the present invention, there is provided an automated behavioural monitoring unit comprising:
(a) one or more sensors;
(b) a processor in the unit connected to the sensors;
(c) an object library stored on the unit; and
(d) a program adapted to:
   i. recognize and classify objects detected by the sensors by accessing the object library stored on the unit;
   ii. track the orientation of the objects with respect to each other;
   iii. identify spatial orientations of the objects and people which are abnormal; and
   iv. communicate details of abnormalities to at least one remote user device,
   wherein no imagery from the sensors is transmitted from the unit to the remote user device in order to safeguard the privacy of people being monitored.

The unit is preferably capable of recognizing a variety of objects using artificial intelligence by reference to the object library. Preferably, the metadata from the processor is transmitted to a remote server. More preferably, no imagery is transmitted from the processor to the remote user devices. The sensors may include infra-red or microwave sensors. The sensors may be remotely controlled moveable sensors. For example, the remotely controlled moveable sensors may be aerial drones.

Any of the features described herein can be combined in any combination with any one or more of the other features described herein within the scope of the invention.

### BRIEF DESCRIPTION OF DRAWINGS

Various embodiments of the invention will be described with reference to the following drawings, in which:

FIG. 1 is a depiction of a device in a room for monitoring the movements of objects and people.

FIG. 2 is a front view of the device of FIG. 1.

FIG. 3 is a flow chart showing the methodology used by the device of FIG. 1 to monitor objects and their interactions.

### DETAILED DESCRIPTION

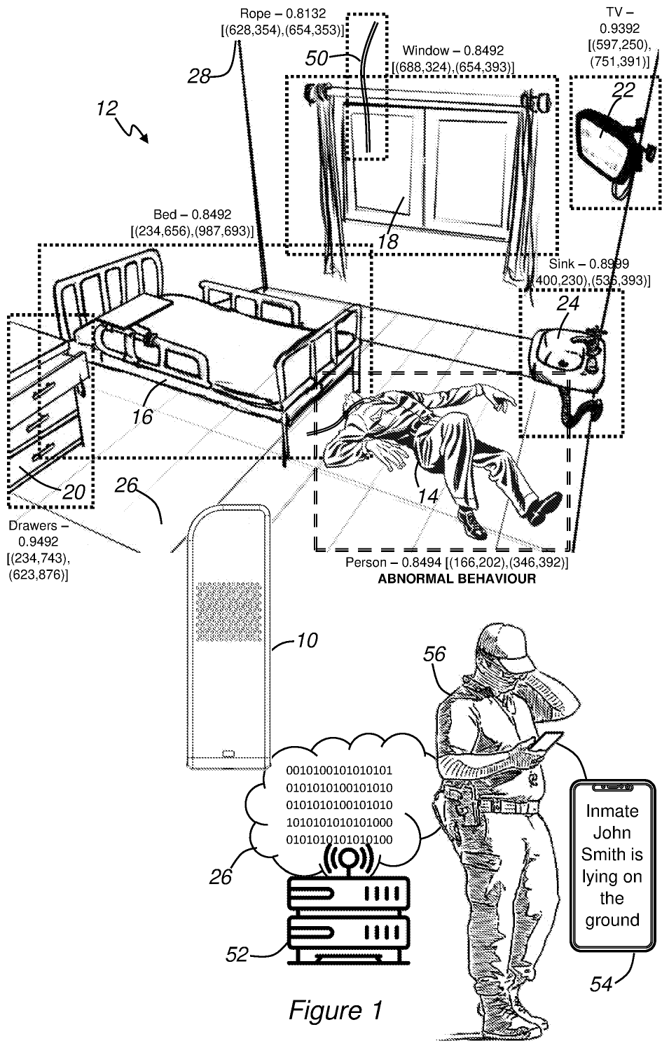

FIG. 1 shows a unit 10 monitoring a room 12. The room 12 is in a prison and the person being monitored is an inmate 14. The room 12 has a bed 16, a window 18, a set of drawers 20, a television 22, a sink 24, a floor 26 and a roof 28.

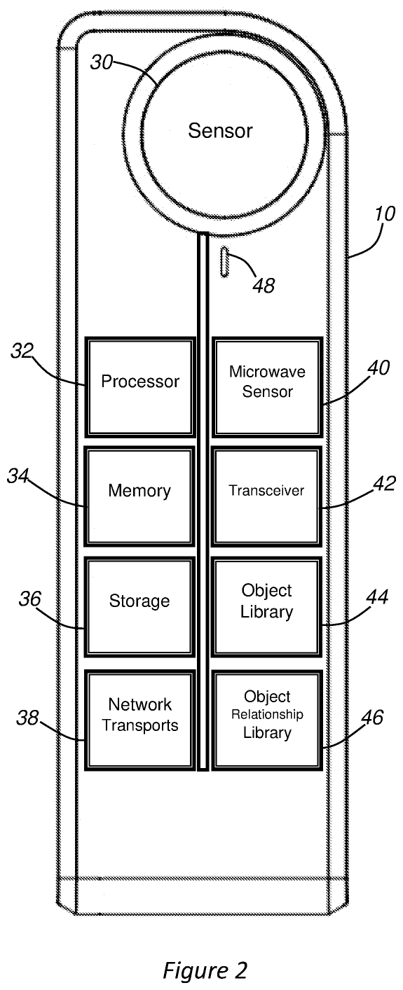

As shown in FIG. 2, the unit 10 that has a sensor 30, a processor 32, a memory 34, hard drive storage 36, network transports 38, a microwave sensor 40, a transceiver 42, an object library 44, an object relationship library 46 and a light indicator 48.

The object library 44 has data of at least one thousand different objects. The objects include for example, various items of furniture, people and phenomena such as fire. The data set is continually revised and updated. Each image was input into the object library manually by the inventors. The nature of each object was categorized in each image.

The unit 10 is capable of recognizing a variety of objects using artificial intelligence by reference the object library. For example, the unit 10 can recognize a new form of chair even though it has not seen that particular type of chair before because that new chair has certain overall characteristics of a chair, such as a seat and a backrest.

A custom set of objects may be created for each individual scenario. For example, one client may want the system to also recognise the uniforms of prison warden staff as well as the uniforms of inmates. Another client may want the system to recognise the outfits of medical staff as well as patient uniforms.

The object library 44 may include sound objects, such as the sound of a person coughing, loud bangs, windows smashing or gun shots. The range of objects is determined by the nature of the sensor 30. For example, the microwave sensor 40 allows the unit 10 to distinguish flesh and blood objects from other inanimate objects. Microwaves are particularly well adapted to go through solid objects include walls and detect people in neighbouring rooms, for example. For example, the microwave sensor 40 can see a person fall in an en suite behind a wall. The sensor 30 is capable of seeing in the visual and infra-red spectrums. The infra-spectrum is particularly useful for seeing objects at night.

The objects are identified by the processor 32 using the sensor 40 by putting boundary lines around each object. The processor 32 translates the visual data from the sensor 30 into imagery metadata, namely coordinates, as shown in FIG. 1.

The unit 10 also includes an object relationship library 46, which tells the processor how any two objects should interact with each other. This includes whether an interaction is normal or abnormal. Each object in the object library 44 was manually categorised as whether its interaction with other objects was normal or abnormal. For example, each object in the library 44 was categorized as having an abnormal relationship with fire.

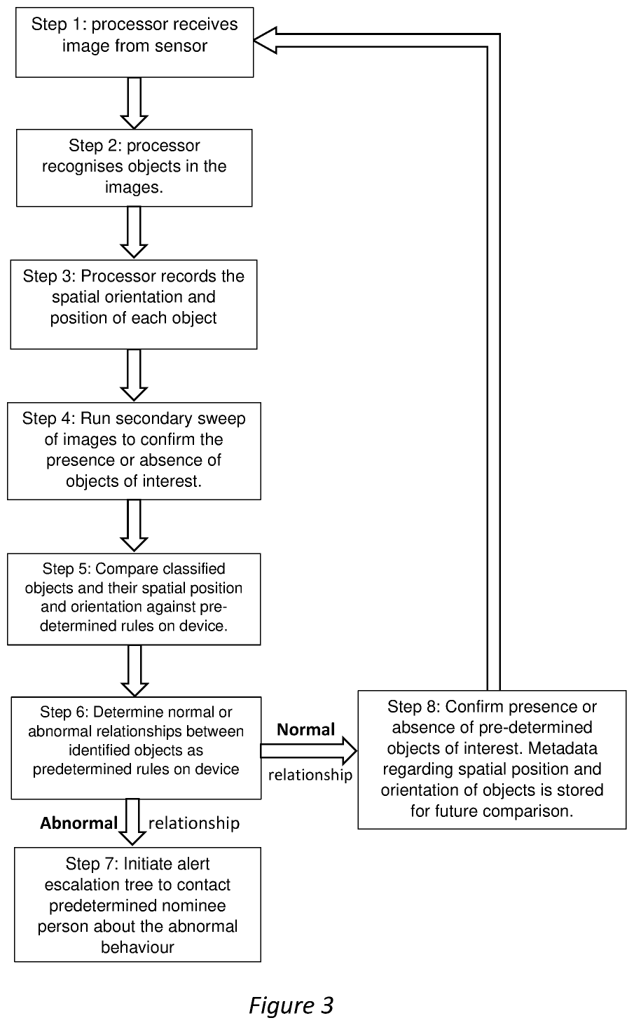

The unit 10 is programmed with an algorithm shown in FIG. 3 to assess objects and their relationship to each other.

In step 1 of the algorithm, the processor 32 receives images from the sensor 30. In step 2, the processor 32 recognises objects in the images using a first artificial intelligence program and classifies them using the object library 44. In step 3, the processor 32 records the spatial orientation of each object. In step 4, the processor 32 runs a secondary sweep of the images to confirm the presence of absence of objects of interest. The secondary sweep of the images is performed by a higher precision artificial intelligence program. The higher precision artificial intelligence program is more resource intensive which takes longer to run. The first image sweep is the human equivalent of glancing at a scene and the secondary check is the equivalent of staring at the scene for confirmation.

In step 5, the processor 32 is programmed to compare classified objects and their spatial position and orientation against pre-determined rules on unit. For example, the processor 32 is programmed to know that the boundary line of the television 22 should not appear over the boundary line of the window 18. This would be an abnormal relationship between the two objects and may indicate, for example, that the inmate 14 is attempting to escape through the window 18 by smashing it with the television 22.

By way of another example, the object library 44 can recognise a rope 50 and the object relationship library 46 can recognise that the rope 50 should not be hanging from the roof 30 of the room 12. In addition, the object library 44 can recognise the person 14 and the object relationship library 46 can recognise that the person 14 should not be lying on the floor 26. This indicates an attempted suicide. In addition, the object relationship library 46 can recognise normal object relationships such as the person 14 lying on the bed 16.

If the processor 32 determines the relationship between two objects is abnormal in step 6 of the algorithm, the processor runs predetermined rules in step 7. The transceiver 42 contacts server 52 via network transport 38 which transmits a message to a remote user device of a predetermined nominee regarding the abnormal behaviour. For example, in the context of FIG. 1, a mobile device 54 of a prison warden 56 receives a message stating: “inmate John Smith is lying on the ground.”

In step 8, the metadata regarding the position and orientation of each object is recorded for future comparison. The processor 32 is programmed not to store or transmit images in order to safeguard the privacy and dignity of the person being monitored. The process is then repeated for the next incoming image from the sensor 30.

### CONCLUDING REMARKS

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. An automated behavioural monitoring unit comprising:
(a) optical and audio sensors;
(b) a processor in the unit connected to the sensors;
(c) an object library stored on the unit; and
(d) a program adapted to:
i. recognize and classify objects detected by the optical and audio sensors by accessing the object library stored on the unit;
ii. track the orientation of the objects with respect to each other;
iii. access an object relationship library which tabulates abnormal interactions between objects in the object library;
iv. visually identify interactions of the objects and people which are abnormal; and
v. communicate details of abnormalities to at least one remote user device,
wherein no imagery from the sensors is transmitted from the unit to the remote user device in order to safeguard the privacy of people being monitored.

2. The automated behavioural monitoring unit of claim 1, wherein the unit is capable of recognizing a variety of objects using artificial intelligence by reference to the object library.

3. The automated behavioural monitoring unit of claim 1, wherein the unit is capable of recognizing a variety of objects programmatically.

4. (canceled)

5. The automated behavioural monitoring unit of claim 1, wherein the optical sensor is an infra-red sensor.
